(19) United States
(12) Plant Patent Application Publication
(10) Pub. No.: US 2003/0046742 P1
(43) Pub. Date: Mar. 6, 2003

(54) Euphorbia plant named "Charam"

(76) Inventor: Bernard Tickner, Bury St. Edmunds (GB)

Correspondence Address: MARK P. BOURGEOIS, P.O. BOX 95, OSCEOLA, IN, 46561, US

(21) Appl. No.: 09/944,932

(22) Filed: Aug. 31, 2001

(51) Int. Cl. A01H 5/00

(52) U.S. Cl. PLT/302

(57) ABSTRACT

A new cultivar of Euphorbia named ‘Charam’ that is distinguished by a compact clumping growth habit, terminal cymes that turn increasingly red in late winter, then open into yellow-green floral heads that completely cover the plant from March through May. In combination with its hardiness and a long period of garden interest, these traits set ‘Charam’ apart from all other existing varieties of Euphorbia known to the inventor.

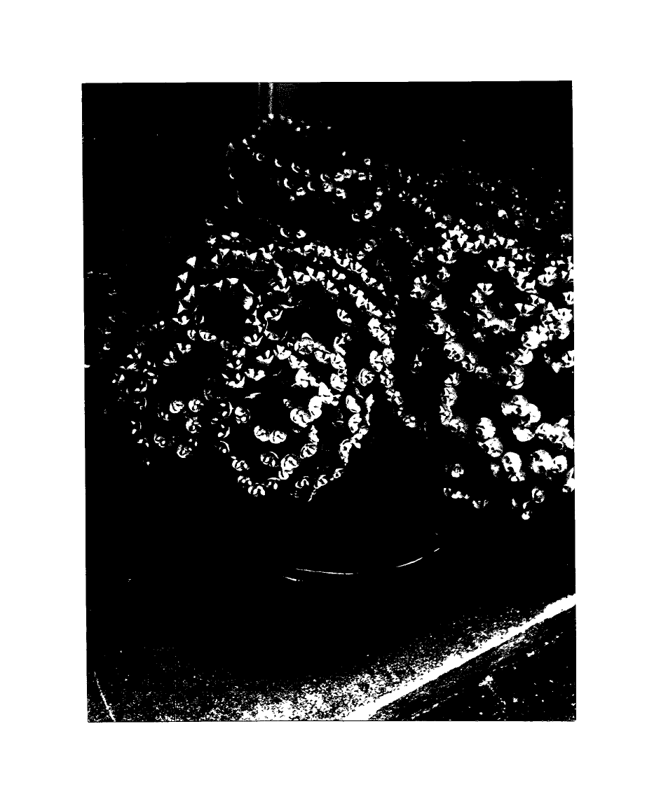

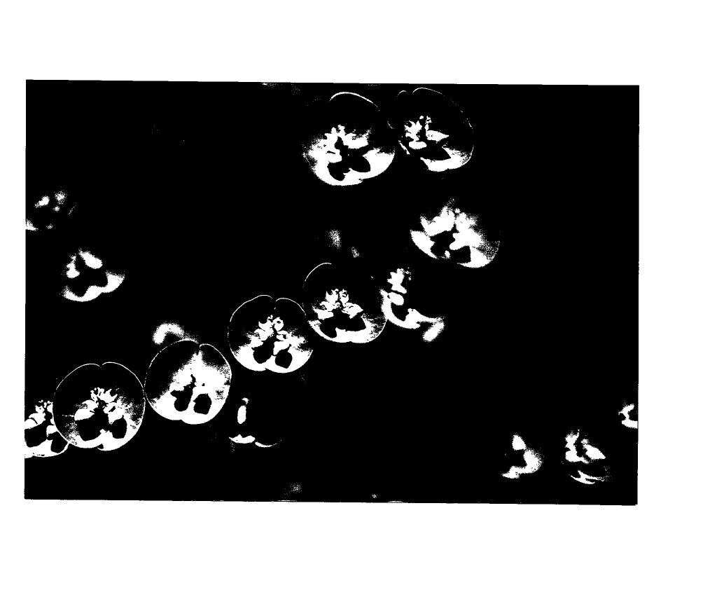

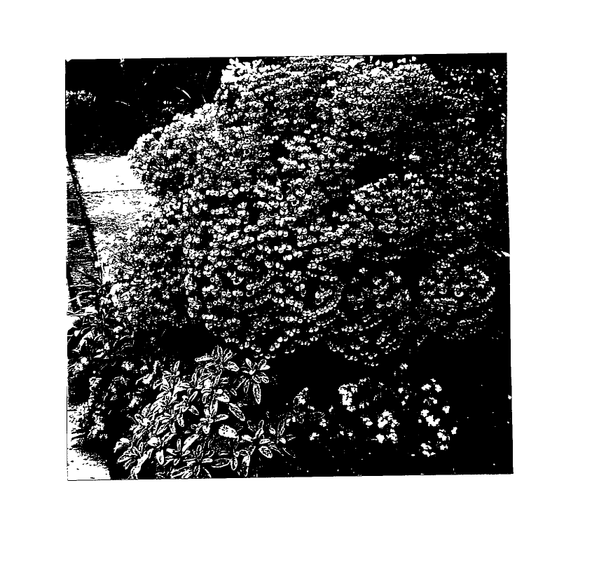

### BACKGROUND OF THE INVENTION

The present invention relates to a new and distinct cultivar of Euphorbia, a hardy perennial that is grown for use as an ornamental landscape plant. The new invention will be referred to hereinafter by the cultivar name ‘Charam’.

‘Charam’ was discovered by the inventor in 1992 in Bury St. Edmunds, England as a hybrid seedling that occurred as the result of a natural cross between the female parent Euphorbia characias sub sp.wulfenii ‘Purple and Gold’ (not patented) and the male parent Euphorbia martinii (not patented) ‘Charam’ is unique and was selected for its compact clumping growth habit, length of garden interest, and terminal cymes that turn increasingly red in late winter, then open to yellow and green in spring forming a ‘hummock’ completely covered by flower color. The flowers are held on terminal stems and born axial to the leaf joints with two cyathia cupped by two basally fused floral leaves, also called involucres.

The closest comparison plants are Euphorbia characias ‘Humpty Dumpty’ (not patented) and Euphorbia×martinii ‘Red Dwarf’ (not patented). ‘Charam’ is distinguishable from both plants by its full floral heads as well as its yellow-green nectary and ovary. The Euphorbias of this group are considered monoecious. In most Euphorbias the stamens mature after the female parts of the flower. However thus far the inventor has not observed stamens and suspects ‘Charam’ to be monoecious with rudimentary stamens that may not be visible to the naked eye.

The first asexual reproduction of ‘Charam’ was accomplished by the inventor using the method of cuttings and was carried out in Bury St. Edmunds, England in 1992. Since that time subsequent generations have been determined stable and true to type.

### SUMMARY OF THE INVENTION

The following traits have been repeatedly observed and represent the distinguishing characteristics of ‘Charam’ . These traits in combination distinguish ‘Charam’ from all other existing varieties of Euphorbia. ‘Charam’ has not been tested under all possible conditions and phenotypic differences may be observed with variations in environmental, climatic, and cultural conditions, however, without any variance in genotype.

1. Euphorbia ‘Charam’ exhibits a compact clumping growth habit.

2. Euphorbia ‘Charam’ exhibits terminal cymes that turn increasingly red in late winter, then open to yellow-green from March through May.

3. Euphorbia ‘Charam’ is evergreen and offers a long period of garden interest.

4. Euphorbia ‘Charam’ is a complete ‘hummock’ of flower color when in full bloom.

5. Euphorbia ‘Charam’ is hardy to minus 12° Centigrade.

6. Euphorbia ‘Charam’ is 70 cm. in height and 70 cm. in width at maturity.

### BRIEF DECRIPTION OF THE DRAWINGS

The accompanying photographs illustrate the distinguishing traits of the new cultivar ‘Charam’. The photographs were taken in spring using plants that were 12 months and grown outdoors in two-gallon containers. The photograph on sheet 1 is taken from a side perspective and illustrates the lower leaves as well as the blooms. The photograph on sheet 2 is a close-up of the inflorescence. The photograph on sheet 3 shows ‘Charam’ in full bloom (the larger plant) planted in the ground with three different small plants in the foreground. The photographs were made using conventional photographic techniques and although flower and foliage colors in the photographs may appear different from actual colors due to light reflectance, they are as accurate as possible by conventional photography.

### BOTANICAL DESCRIPTION OF THE PLANT

The following is a detailed botanical description of the new Euphorbia cultivar ‘Charam’. Observations, measurements, values and comparisons have been collected in spring in Arroyo Grande, Calif. from plants that were 12 months and grown outdoors in two-gallon containers. Color determinations are made in accordance with The Royal Horticultural Society Colour Chart from London England, except where general color terms of ordinary dictionary significance are used. The growing requirements of the new variety are similar to the species and there are no known growing problems, diseases or pests.

Botanical classification: Euphorbia ‘Charam’.

Common name: Spurge.

Parentage: Euphorbia ‘Charam’ is a natural seedling that resulted from the spontaneous hybridization of the following parents:

Female parent: Euphorbia characias sub sp.wulfrenii ‘Purple and Gold’.

Male parent: Euphorbia martinii. Propagation method: Terminal stem cuttings Rooting habit: Fibrous rooting habit Time to develop roots: In California 2-3 weeks are required for roots to develop on an initial cutting Crop time: In California 6-8 months are required to develop a finished one-gallon container from a rooted cutting Growth habit: Upright, compact and clumping growth habit Use: Ornamental landscape plant Type: Perennial herb Vigor: Vigorous Height of plant: 50-70 cm. in height Width of plant: 50-70 cm. in width Sexuality: ‘Charam’ is assumed to be monoecious with rudimentary stamens that mature after the female parts. Cultural requirements: Plant in full sun and well-drained soil Hardiness: Hardy to minus 12° Centigrade. Stem:

Branching habit: Upright.

Trunk dimensions: 2 cm. in diameter and 3 cm. from soil level to first branching.

Stem color: Mostly 144A with streaks of 59A.

Stem shape: Cylindrical to columnar.

Stem width: .75 cm. in diameter.

Stem length: 30 cm. in length.

Internode length: .50 cm. between nodes.

Stem surface: Mostly smooth with some pubescence. Basal surface to mid-stem is heavily covered with bundle scars. Some terminal stem surfaces are rugose.

Bundle scars: Present on stem surface.

Shape of bundle scars: Linear shaped.

Dimensions of bundle scars: 3 mm. in length and 1 mm. in width.

Color of bundle scars: 199D.

Quantity of bundle scars: Numerous amounting to approximately 42 on a 20 cm. long stem.

Stem texture: Fleshy and flexible.

Color of pubescence: 198D.

Other.—Plant parts exude a white milky sap that can be toxic and can ellicit dermal irritation.

Foliage:

Type.—Evergreen.

Leaf arrangement.—Whorled.

Leaf division.—Simple.

Leaf shape.—Oblanceolate to spatulate.

Mature leaf length.—9 cm. in length.

Mature leaf width.—1 cm. in width.

Young leaf length.—2.5 cm. in length.

Young leaf width.—.75 cm. in width.

Internode length.—1 cm. between nodes.

Leaf apex.—Acuminate.

Leaf base.—Truncate.

Quantity of leaves.—Numerous amounting to approximately 25 leaves on a 7 cm. long stem.

Leaf venation pattern.—Pinnate pattern with a barely visible mid-vein on the adaxial surface and a prominent protruding mid-vein on the abaxial surface.

Vein color (adaxial surface).—138B.

Vein color (abaxial surface).—59A.

Margin.—Entire.

Leaf surface (abaxial surface).—Pubescent.

Leaf surface (adaxial surface).—Sparsely puberulent.

Color of pubescence.—198D.

Leaf attachment.—Sessile.

Mature leaf color (adaxial surface).—137A.

Mature leaf color (abaxial surface).—138A.

Young leaf color (adaxial surface).—Mostly 137C and tinged with 59A at margins.

Young leaf color (abaxial surface).—Mostly 137D and tinged with 59A towards the apex.

Flower:

Inflorescence.—Terminal cyme .

Dimensions of inflorescence.—7 cm. in length and 4 cm. in width.

Flowering season.—Late winter until late spring.

Peduncle dimensions.—15 cm in length and 2 mm. in width.

Peduncle color.—Mostly 144A with tinge of 59A.

Peduncle surface.—Sparsely puberulent.

Color of pubescence.—198D.

Petals.—Apetalous.

Sepals.—Asepalous.

Floral leaf attachment.—Connate perfoliate.

Dimensions of floral leaves.—12 mm. in length and 2 cm. in width.

Shape of floral leaf.—Closest to reniform.

Color of floral leaf.—144B with streaks of 151B.

Number of floral leaves.—Two in number.

Fused or unfused.—Basally fused.

Internode length.—5-6 cm. between nodes.

Number of cyathium.—Two cupped within the center of two basally fused floral leaves.

Shape of cyathium.—Cupulate.

Number of floral leaves on each cyathium.—Two.

Fused or unfused.—Basally fused.

Shape of floral leaves on cyathia.—Closest to reniform.

Color of cyathium.—144C.

Dimensions of cyathium.—5 mm. in length and 6 mm. in width.

Nectary.—Four flattened glands on four fused bracts.

Shape of nectary.—Flattened crescent shaped glands with two horns.

Dimensions of nectary.—6 mm. in height and 6 mm. in diameter.

Color of mature nectary.—161A.

Color of young nectary.—144A.

Position of nectaries.—One green nectary in the center of each cyathium and one yellow nectary axillary to the floral leaf and basal to each pair of cyathia.

Reproductive organs:

Stamens.—Six rudimentary stamens that are visible with a lens after the female parts Have matured.

Stamen color.—200A.

Stamen dimensions.—Less than .25 mm. in length and less than .25 mm. in diameter.

Anther shape.—Oval to round.

Anther color.—200A.

Ovary dimensions.—1.5 mm. in width and 1.5 mm. in length.

Shape of ovary.—Globose.

Position of ovary.—Superior.

Color of ovary.—144A.

Pollen.—Absent.

Pistil.—One in number.

Color of pistil.—144A.

Dimensions of pistil.—7 mm. length and less than 1 mm. in width.

Style.—Three in number:

Style shape.—Bifid:

Style color.—144A.

Style dimensions.—Less than 1 mm. in width and 1 mm. in height.

Stigma.—Six in number.

Stigma color.—144A

Dimensions of Stigma.—Less than .75 mm. in width and less than .75 mm. in height.

Seed:

Seed has not been observed.—

1. A new and distinct cultivar of euphorbia plant named ‘charam’ as described and illustrated: 5 10 1 5 20 25 30 35
